(19) United States
(12) Patent Application Publication
(10) Pub. No.: US 2024/0388777 A1
(43) Pub. Date: Nov. 21, 2024

(54) IMAGING FOR OPTICAL VIEWING DEVICES

(71) Applicant: Welch Allyn, Inc., Skaneateles Falls, NY (US)

(72) Inventors: David G. Perkins, Tully, NY (US); Danielle Endres, Liverpool, NY (US); Lei Guo, Manlius, NY (US); Joshua Hess, Moravia, NY (US); David Villarreal, Fayetteville, NY (US)

(21) Appl. No.: 18/658,152

(22) Filed: May 8, 2024

Related U.S. Application Data
(60) Provisional application No. 63/503,219, filed on May 19, 2023.

(51) Int. Cl. H04N 23/51; G02B 25/00; H04N 23/55; H04N 23/57; H04N 23/60; H04N 23/63; H04N 23/69

(52) U.S. Cl. CPC H04N 23/51; G02B 25/001; H04N 23/55; H04N 23/57; H04N 23/63; H04N 23/64; H04N 23/69

(57) ABSTRACT

An imaging device for capturing images viewed from an optical viewing device. The imaging device includes a housing for attachment to the optical viewing device. The imaging device detects attachment to the optical viewing device. The imaging device determines a type of the optical viewing device. The imaging device adjusts at least one aspect of the imaging device based on the type of the optical viewing device.

### BACKGROUND

Optical viewing devices are used for examining patients as part of routine examinations. Examples of optical viewing devices can include, without limitation, an otoscope for assessing the ears of a patient, an ophthalmoscope for assessing the eyes of a patient, and a dermatoscope for assessing the skin of a patient.

Each optical viewing device has different optical features such as field of view, magnification, illumination, brightness, and color temperature. When used with an imaging device for recording images, each optical viewing device requires a unique camera setting such as digital zoom for optimal results. For example, otoscopes typically have a smaller field of view than other types of optical viewing devices, which requires an imaging device when used on an otoscope to have a higher digital zoom. The higher zoom settings on the imaging device can cause images to move around a display screen in an unstable manner.

### SUMMARY

In general terms, the present disclosure relates to imaging for optical viewing devices. In one possible configuration, an imaging device automatically optimizes at least one aspect for capturing and/or displaying images from an optical viewing device. In another possible configuration, the imaging device displays a diopter value selected on the optical viewing device. Various aspects are described in this disclosure, which include, but are not limited to, the following aspects.

One aspect relates to an imaging device for capturing images viewed from an optical viewing device, the imaging device comprising: a housing for attachment to the optical viewing device; at least one processing device housed inside the housing; and at least one computer-readable data storage device storing software instructions that, when executed by the at least one processing device, cause the at least one processing device to: detect attachment to the optical viewing device; determine a type of the optical viewing device; and adjust at least one aspect of the imaging device based on the type of the optical viewing device.

Another aspect relates to a method of capturing images from an optical viewing device, the method comprising: detecting attachment to the optical viewing device; determining a type of the optical viewing device; and adjusting at least one aspect based on the type of the optical viewing device.

Another aspect relates to an imaging device for capturing images viewed from an optical viewing device, the imaging device comprising: a housing having a bracket for attaching the imaging device to the optical viewing device; a camera for capturing the images through an eyepiece of the optical viewing device; a display screen for displaying the images captured by the camera; at least one processing device; and at least one computer-readable data storage device storing software instructions that, when executed by the at least one processing device, cause the at least one processing device to: display a diopter value on the display screen, the diopter value selected on the optical viewing device.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 shows examples of different types of optical viewing devices, each optical viewing device is shown attached to an imaging device.

FIG. 2 is an isometric view of an example of an optical viewing device shown in FIG. 1, the optical viewing device shown from a physician perspective.

FIG. 3 is another isometric view of the optical viewing device of FIG. 2, the optical viewing device shown from a patient perspective.

FIG. 4 is an isometric view of an example of the imaging device attached to the optical viewing device of FIG. 2, the imaging device shown from the physician perspective.

FIG. 5 is a front isometric view of the imaging device of FIG. 4.

FIG. 6A is a front view of the imaging device of FIG. 4.

FIG. 6B is a rear view of the imaging device of FIG. 4.

FIG. 6C is a top view of the imaging device of FIG. 4.

FIG. 7 is an isometric view showing a camera of the imaging device of FIG. 4.

FIG. 8 is an isometric view of another example of the imaging device attached to the optical viewing device of FIG. 2, the imaging device shown from the physician perspective.

FIG. 9 is an isometric view of the imaging device of FIG. 8 before attachment to the optical viewing device of FIG. 2, the imaging device shown from the patient perspective.

FIG. 10 is an isometric view of a charging station for charging the optical viewing devices of FIG. 1.

FIG. 11 schematically illustrates an example of a method of optimizing at least one feature of the imaging device of FIGS. 1-9 based on the type of the optical viewing device attached to the imaging device.

FIG. 12 is a rear isometric view of another example of the imaging device attached to the optical viewing device of FIG. 2.

FIG. 13 is a front isometric view of the imaging device of FIG. 12 attached to the optical viewing device of FIG. 2.

FIG. 14 is a cross-sectional view of an example of a periscope installed on the imaging device of FIG. 12.

FIG. 15 is a cross-sectional view of another example of a periscope installed on the imaging device of FIG. 12.

FIG. 16 illustrates an exemplary architecture of a computing device of the imaging device shown in any of the above figures.

### DETAILED DESCRIPTION

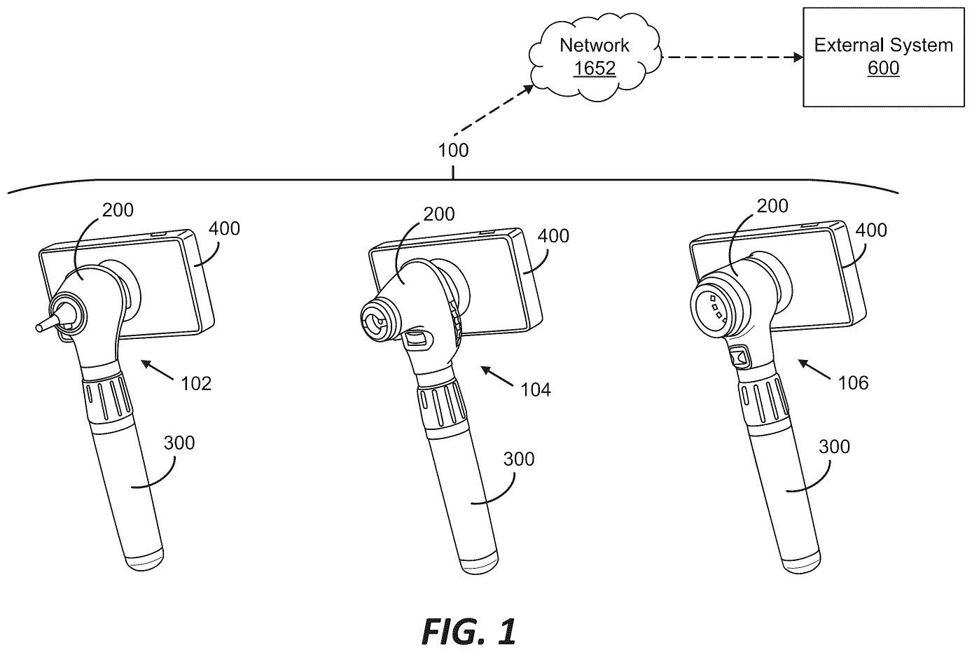

FIG. 1 shows examples of different types of optical viewing devices 100. For example, the optical viewing devices 100 include a first type of optical viewing device 102 such as an otoscope, a second type of optical viewing device 104 such as an ophthalmoscope, and a third type of optical viewing device 106 such as a dermatoscope. Additional types of the optical viewing devices 100 are possible, and the disclosure provided herein is not limited to otoscopes, ophthalmoscopes, and dermatoscopes.

As shown in FIG. 1, each type of optical viewing device 100 includes an instrument head 200 attached to an instrument handle 300. The instrument head 200 can include a light source and optics for viewing an anatomical area of interest through an eyepiece. The instrument handle 300 can include a power source that powers the light source and other components of the instrument head 200. For example, the instrument handle 300 can include rechargeable batteries, disposable batteries, or a tether to a wall transformer for supplying electrical power to the components of the instrument head 200.

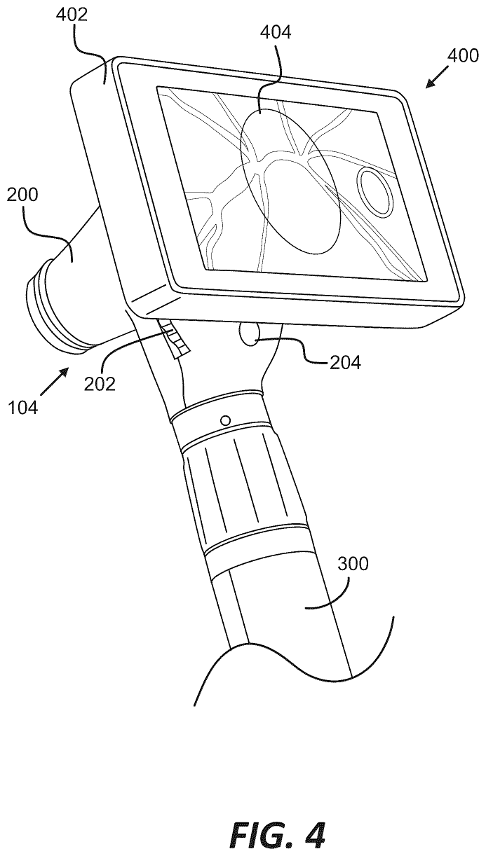

As further shown in FIG. 1, an imaging device 400 is attached to the instrument head 200 of each type of optical viewing device 100. The imaging device 400 is a portable, battery powered camera that can record high quality image frames and videos from the optical viewing devices 100, providing digital imaging solutions. For example, the imaging device 400 captures images through an eyepiece of the instrument head 200 for display on a display screen 404 (see FIG. 4) for viewing by a physician. The images captured by the imaging device 400 can be analyzed by algorithms (including artificial intelligence algorithms) for disease screening, and the images can be stored in an electronic medical record (EMR) of a patient.

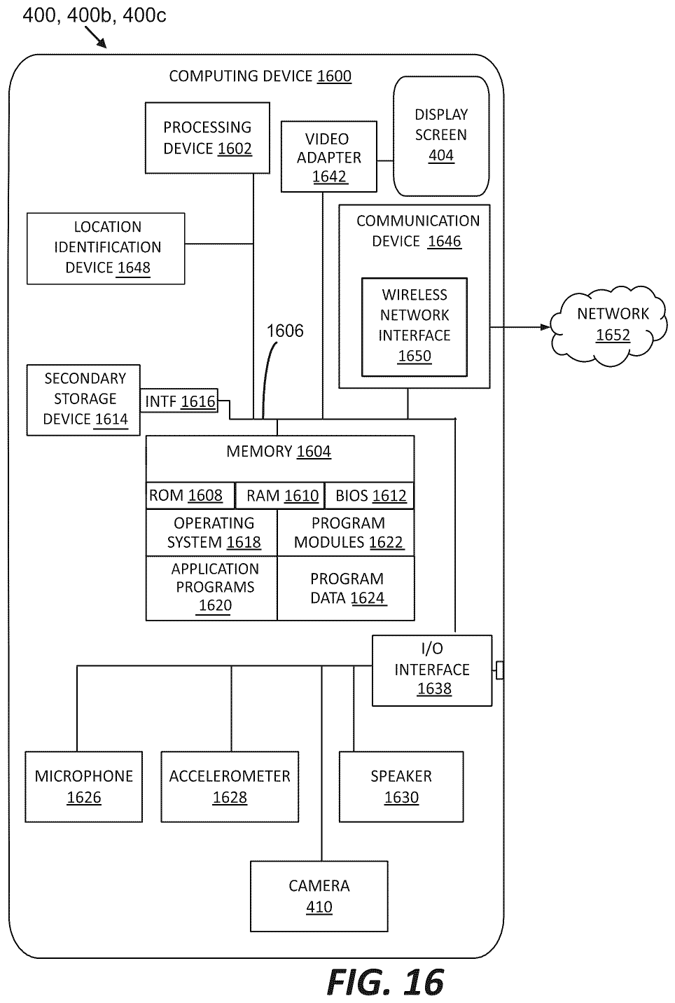

In some examples, the imaging device 400 transmits images, videos, and other data to an external system 600, which analyzes the images, videos, and other data to generate one or more results for transmission back to the imaging device 400. The external system 600 can be remotely located with respect to the optical viewing device 100 and the imaging device 400. In some examples, the external system 600 includes a cloud server. The imaging device 400 can communicate with the external system 600 via a network 1652 (see also FIG. 16).

The algorithms (including artificial intelligence algorithms) for disease screening can be executed on either or both of the imaging device 400 and the external system 600. In some examples, the external system 600 may also host storage of the images, videos, and other data received from the imaging device 400. In further examples, the external system 600 can host the EMR of the patient. In yet further examples, the external system 600 may provide connectivity to other external systems and servers having image storage, or that host the EMR.

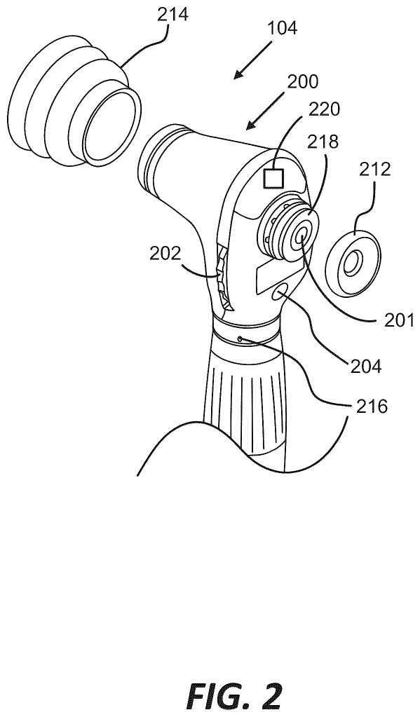

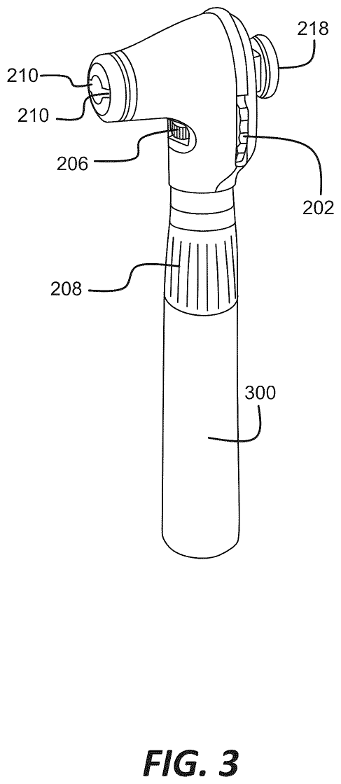

FIGS. 2 and 3 are isometric views of an example of the second type of optical viewing device 104 (i.e., ophthalmoscope). In FIG. 2, the second type of optical viewing device 104 is shown from a physician perspective. In FIG. 3, the second type of optical viewing device 104 is shown from a patient perspective. While FIGS. 2 and 3 refer to the second type of optical viewing device 104, the first type of optical viewing device 102 and the third type of optical viewing device 106 can include similar components and features.

Referring now to FIGS. 2 and 3, the second type of optical viewing device 104 includes a diopter focus wheel 202 and a diopter readout 204. The diopter focus wheel 202 can be used to adjust a focus of an eyepiece 201. For example, the diopter focus wheel 202 can be used to correct the refractive errors of both the user of the optical viewing device 104 and the patient. For example, the diopter focus wheel 202 can be used to provide a positive dioptric value to accommodate for hyperopia eyesight (farsightedness) of both the user of the optical viewing device 104 and the patient, and to provide a negative dioptric value to accommodate for myopia eyesight (nearsightedness) of both the user of the optical viewing device 104 and the patient.

The dioptric values adjusted by using the diopter focus wheel 202 are displayed in the diopter readout 204. In some examples, positive dioptric values are displayed in the diopter readout 204 in a first color (e.g., green), and negative dioptric values are displayed in the diopter readout 204 in a second color (e.g., red). In some instances, the diopter readout 204 can be obscured by the imaging device 400 when attached to the instrument head 200.

The second type of optical viewing device 104 can further include a filter wheel 206 to select a filter for viewing through the eyepiece 201. For example, the filter wheel 206 can be used to select a reticle target to measure the optic disc, a cobalt blue filter to detect corneal abrasions, a red-free filter, and additional types of filters.

The second type of optical viewing device 104 can further include a light control 208 for controlling illumination from the light source, disc alignment lights 210 (e.g., red for right eye exams; yellow for left eye exams), an eyepiece bumper 212, an optional patient eye cup 214, an optional locking collar 216, and an eyepiece housing 218. As will be described in more detail below, the imaging device 400 includes a bracket that removably attaches to the eyepiece housing 218 for securing the imaging device 400 to the instrument head 200.

As further shown in FIG. 2, the second type of optical viewing device 104 can include an identifier 220. While the identifier 220 is described with reference to the second type of optical viewing device 104, the first and third types of optical viewing devices 102, 106, as well as additional types of optical viewing devices can similarly include the identifier 220, as described herein. As will be described in more detail, the identifier 220 provides machine-readable data that can be detected by the imaging device 400 to detect attachment of the imaging device 400 to the instrument head 200, and to convey additional information such as the type of the instrument head 200 (i.e., whether the instrument head 200 is for an otoscope, ophthalmoscope, dermatoscope, or other type of optical viewing device).

In some examples, the identifier 220 is a wireless antenna that transmits a wireless signal that is detected by the imaging device 400 when the imaging device 400 is attached to the instrument head 200. In some examples, the wireless signal transmitted by the identifier 220 to the imaging device 400 can include a radio frequency identification (RFID) signal, a near field communication (NFC) signal, a Bluetooth signal, a Wi-Fi signal, or other similar wireless signals. The identifier 220 can include a passive antenna or tag that is activated by an active antenna on the imaging device 400 to transmit the wireless signal when the imaging device 400 is in close proximity to the instrument head 200 such as when it is attached thereto.

In some further examples, the identifier 220 can provide additional types of machine-readable data that can be detected by the imaging device 400. For example, the identifier 220 can include a quick response (QR) code or other type of machine-readable label that can be read by a primary camera or a secondary camera of the imaging device 400.

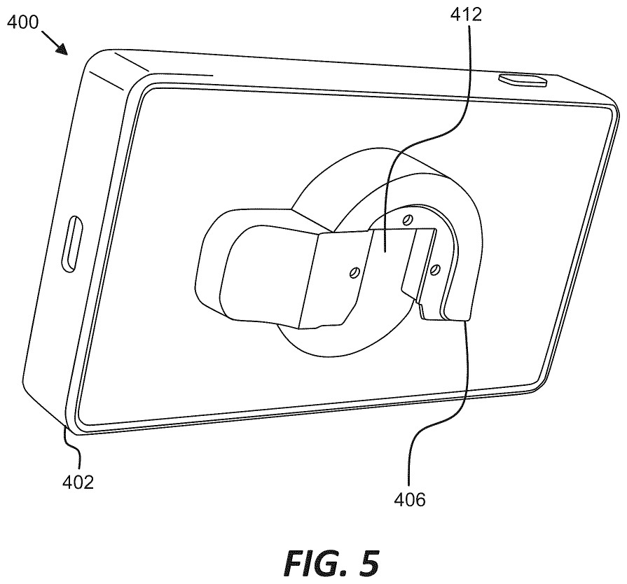

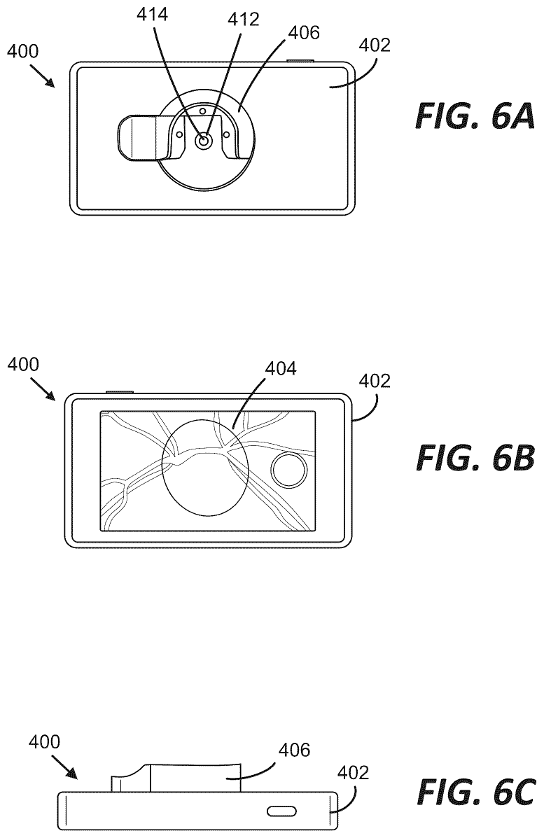

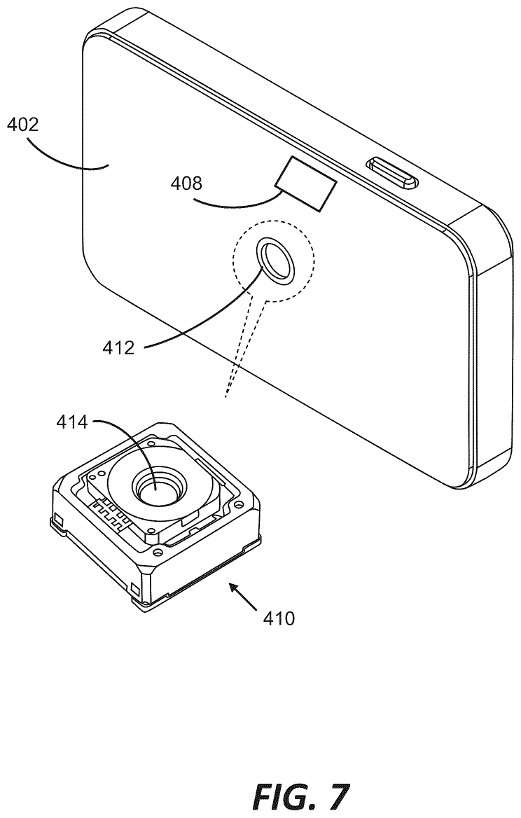

FIG. 4 is an isometric view of an example of the imaging device 400 attached to the second type of optical viewing device 104. In FIG. 4, the imaging device 400 shown from the physician perspective. FIG. 5 is a front isometric view of the imaging device 400. FIG. 6A is a front view of the imaging device 400. FIG. 6B is a rear view of the imaging device 400. FIG. 6C is a top view of the imaging device 400. FIG. 7 is an isometric view showing a camera 410 of the imaging device 400. Referring now to FIGS. 4-7, the imaging device 400 captures images viewed from the eyepiece 201 of an optical viewing device 100. While FIGS. 4-6 show the imaging device 400 attached to the second type of optical viewing device 104, the imaging device 400 can be similarly attached to the first and third types of optical viewing devices 102, 106 for capturing images viewed from the eyepieces 201 of those devices.

As shown in FIGS. 4-6, the imaging device 400 includes a housing 402. In this example, a bracket 406 is integrated with a back surface of the housing 402. The bracket 406 allows the imaging device 400 to physically attach to the optical viewing devices 100. For example, the bracket 406 can be fixed around an eyepiece housing 218 (see FIGS. 2 and 3) for attaching the imaging device 400 to an instrument head 200. In alternative examples, the bracket 406 can be part of an accessory case that attaches to the housing 402, and that can be used to physically attach the imaging device 400 to the optical viewing devices 100.

As shown in FIG. 7, the housing 402 further includes an aperture 412 for a lens 414 of the camera 410. The camera 410 is mounted inside the housing 402 of the imaging device 400. When the imaging device 400 is mounted to the instrument head 200, the camera 410 is aligned with the eyepiece 201 of the instrument head 200 for capturing images viewed through the eyepiece 201 of the instrument head 200. The camera 410 is centrally mounted inside the housing 402 to provide even balance and weight distribution for when the imaging device 400 is attached to the instrument head 200, thereby improve the ergonomics of the assembly. A protrusion of the lens 414 beyond the back surface of the housing 402 is minimized such that the lens 414 is substantially flush with the back surface of the housing 402.

The camera 410 can include features such as auto focus, auto-exposure, auto white-balance, and image stabilization. The camera 410 can include a 12MP color image sensor. As an illustrative example, the camera 410 can include an equivalent focal length (on 35 mm film) of 52-77 mm, 4K (30FPS) video recording with 4000×3000 pixel resolution, and a record time of 90 minutes at 4K resolution, 1 minute per clip. Alternative camera parameters are possible.

The housing 402 is compact and lightweight. In some examples, the housing 402 includes a protective overmold having a base layer of plastic material, and a top layer of rubber to provide shock absorption and improved grip. The housing 402 can include one or more ports such as a USB-C port for charging the battery, and for data transferring including uploading images and videos captured by the camera 410 to another device. As an illustrative example, the housing 402 can have a thickness (e.g., distance between the lens 414 of the camera 410 and the display screen 404) that is less than 25 mm, and a weight that is less than 250 g. The housing 402 can include a power button to turn on/off and wake up the imaging device 400. The housing 402 houses an integrated, rechargeable battery that can, for example, power 90 minutes of 4K video recording by the camera 410, and 3-4 hours of screen time on the display screen 404.

As shown in FIG. 7, the imaging device 400 can include a detector 408 that detects the machine-readable data from the identifier 220 on the instrument head 200 (see FIG. 2) to detect attachment of the imaging device 400 to the instrument head 200, and to detect additional information such as the type of the instrument head 200 (i.e., whether the instrument head 200 is for an otoscope, ophthalmoscope, dermatoscope, or other type of optical viewing device).

In some examples, the detector 408 is a wireless antenna that detects a wireless signal from the identifier 220 on the instrument head 200 when the imaging device 400 is attached to the instrument head 200. In some examples, the detector 408 on the imaging device 400 can detect a radio frequency identification (RFID) signal, a near field communication (NFC) signal, a Bluetooth signal, a Wi-Fi signal, or other similar wireless signals emitted from the instrument head 200. The detector 408 can include an active antenna or tag that activates a passive antenna or tag on the instrument head 200 to receive a transmission of the wireless signal. In some examples, the active antenna is mounted on the imaging device 400 in a location that corresponds to the placement of the passive antenna on the instrument head 200 such that the active antenna activates the passive antenna when in close proximity to the passive antenna such as when the imaging device 400 is attached to the instrument head 200.

In some further examples, the detector 408 can include a secondary camera that can read a quick response (QR) code or other type of machine-readable label placed on the instrument head 200. The secondary camera can read the machine-readable label to detect attachment of the imaging device 400 to the instrument head 200, as well as to determine the type of the instrument head 200 (i.e., whether the instrument head 200 is for an otoscope, ophthalmoscope, dermatoscope, or other type of optical viewing device). The secondary camera can be mounted on the imaging device 400 in a location that corresponds to the placement of the machine-readable label on the instrument head 200.

As further shown in FIGS. 4-7, the imaging device 400 includes a display screen 404 for displaying the images captured by the camera 410. In some examples, the display screen 404 is a touchscreen such that it can both display the images, and receive inputs from a user. For example, the display screen 404 can be used by a user of the imaging device to: adjust the settings of the camera 410 (e.g., focus, exposure, white balance, FOV/zoom); tapping the display screen 404 to trigger focus and lock; adjust settings of the display screen 404 such as the screen brightness; provide a virtual keyboard to type in information; display a battery-life indicator; provide video recording controls (e.g., start, stop, save, delete, review, upload; provide a sliding bar to go through video frames, pinch-zoom to enlarge; display arrow(s) to indicate image orientation; and display one or more stamps (e.g., date, time, filter info, etc.) on saved images.

The display screen 404 can include a true color multi-touch screen (in-plane switching (IPS), or light-emitting diode (LED)). The display screen 404 can have a bezel-less design (e.g., full-screen display). The display screen 404 can have a resolution of at least 250 pixels per inch (PPI), a diagonal screen size of about 2 inches to about 5 inches, an aspect ratio of 16:9/4:3, a maximum brightness of 500 nits. The display screen 404 can also include features such as screen auto off, and wake up by power button or tapping the display screen 404.

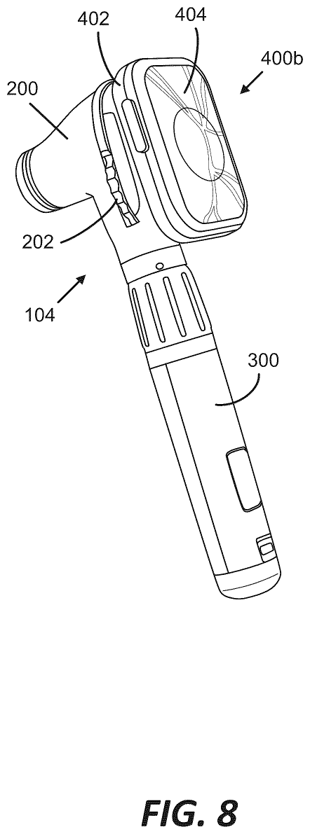

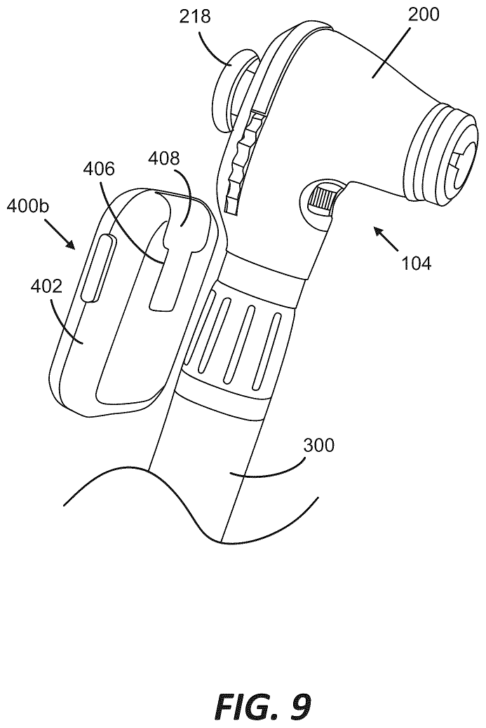

FIG. 8 is an isometric view of another example of an imaging device 400b attached to the second type of optical viewing device 104. In FIG. 8, the imaging device 400b is shown from the physician perspective. FIG. 9 is an isometric view of the imaging device 400b before attachment to the second type of optical viewing device 104. In FIG. 9, the imaging device 400b is shown from the patient perspective. While FIGS. 8 and 9 show the imaging device 400b attached to the second type of optical viewing device 104, the imaging device 400b can similarly attach to the first and third types of optical viewing devices 102, 106, and to additional types of optical viewing devices for capturing and displaying images.

The imaging device 400b is similar to the imaging device 400 shown in FIGS. 4-7. For example, the imaging device 400b includes a housing 402 having a bracket 406 for attaching to the eyepiece housing 218 of the optical viewing device 100. The imaging device 400b similarly includes a display screen 404 for displaying images captured by a camera that is centrally mounted inside the housing 402 of the imaging device 400b to provide even balance and weight distribution. Like in the examples described in FIGS. 4-7, the camera of the imaging device 400b is configured for alignment with the eyepiece 201 of the instrument head 200 for capturing images viewed through the eyepiece 201 of the instrument head 200.

The imaging device 400b can also include a detector 408 for detecting machine-readable data from the instrument head 200 such as to detect attachment of the imaging device 400b to the instrument head 200, and to detect additional information from the instrument head 200 such as the type of the instrument head 200 (i.e., whether the instrument head 200 is for an otoscope, ophthalmoscope, dermatoscope, or other type of optical viewing device).

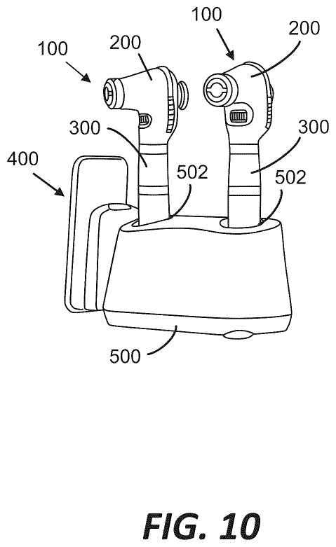

FIG. 10 is an isometric view of a charging station 500 for charging the optical viewing devices 100. For example, each instrument handle 300 can be inserted into an aperture 502 of the charging station 500 for charging the power source in the instrument handle 300 when the optical viewing device 100 is not being used. As further shown in FIG. 10, the imaging device 400 can also be held on the charging station 500 for storage and charging.

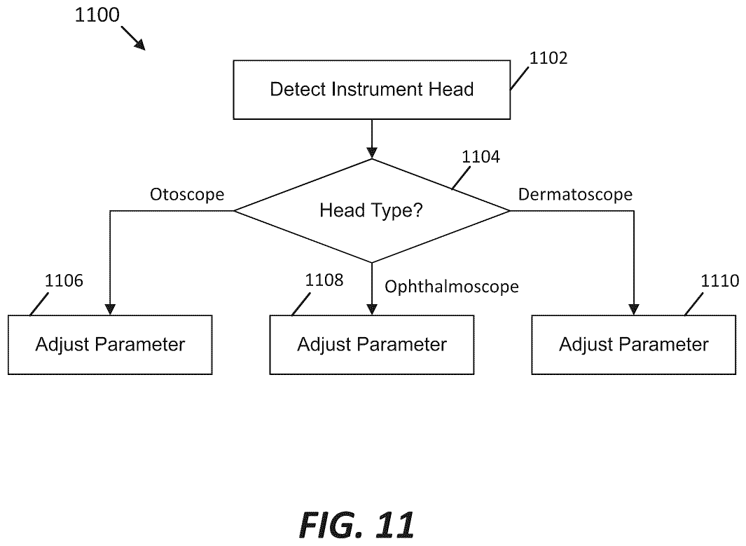

FIG. 11 schematically illustrates an example of a method 1100 of optimizing at least one feature of the imaging device 400, 400b based on the type of the optical viewing device 100 attached to the imaging device 400, 400b. As described above, the imaging device 400, 400b can attach to the first type of optical viewing device 102 such as an otoscope, to the second type of optical viewing device 104 such as an ophthalmoscope, to the third type of optical viewing device 106 such as a dermatoscope, and to additional types of optical viewing devices. The method 1100 is automatically performed by the imaging device 400, 400b without requiring any input or feedback from a user, thereby improving the usability of the imaging device 400, 400b by having one or more features of the imaging device 400, 400b automatically adjusted based on the type of optical viewing device attached thereto.

As shown in FIG. 11, the method 1100 includes an operation 1102 of detecting attachment to the instrument head 200. As described above, the imaging device 400, 400b attaches to the eyepiece housing 218 via the bracket 406.

Operation 1102 can include detecting attachment to the instrument head 200 based on the images captured by the imaging device 400, 400b. For example, attachment to an otoscope is detected when the imaging device 400, 400b detects images of an ear anatomy. As a further example, attachment to an ophthalmoscope is detected when the imaging device 400, 400b detects images of an eye anatomy. As a further example, attachment to a dermatoscope is detected when the imaging device 400, 400b detects images of a skin anatomy.

In further examples, operation 1102 can include detecting attachment to the instrument head 200 based on physical contact with the instrument head 200. For example, the imaging device 400, 400b can include a strain gauge or similar type of sensor inside the bracket 406 that can detect physical contact between the bracket 406 and the eyepiece housing 218.

In further examples, operation 1102 can include detecting attachment to the instrument head 200 based on an electrical connection between the imaging device 400, 400b and the instrument head 200. For example, the imaging device 400, 400b can include one or more electrical contacts that complete a circuit when in contact with one or more electrical contacts on the instrument head 200. In such examples, operation 1102 includes detecting attachment to the instrument head 200 when the one or more electrical contacts on the bracket 406 complete the circuit with the one or more electrical contacts on the instrument head 200.

In further examples, operation 1102 can include detecting attachment to the instrument head 200 based on machine-readable data provided by the identifier 220 on the instrument head 200. As described above, in some examples, the imaging device 400, 400b can include a detector 408 that reads the machine-readable data from the instrument head 200.

In some examples, operation 1102 can include detecting attachment of the imaging device 400, 400b to the instrument head 200 based on a wireless signal received from the instrument head 200. For example, the instrument head 200 can include a wireless antenna that transmits a wireless signal that can be picked up by a wireless antenna on the imaging device 400, 400b when the imaging device is attached to the instrument head 200. As an illustrative example, the wireless signal transmitted from the instrument head 200 to the imaging device 400, 400b can include a radio frequency identification (RFID) signal, a near field communication (NFC) signal, a Bluetooth signal, a Wi-Fi signal, or other similar wireless signals.

In some examples, the wireless antenna on the instrument head 200 is a passive antenna and the wireless antenna on the imaging device 400, 400b is an active antenna such that the wireless antenna on the instrument head 200 does not transmit the wireless signal unless activated by the wireless antenna on the imaging device 400, 400b such as when the imaging device 400, 400b is attached to the instrument head 200. In some examples, the wireless antenna on the instrument head 200 is an RFID tag, an NFC tag, or similar type of wireless signal tag.

In further examples, operation 1102 can include detecting attachment to the instrument head 200 by reading a quick response (QR) code or other similar type of machine-readable label on the instrument head 200. For example, operation 1102 can include using the camera 410 of the imaging device 400, 400b to read a machine-readable label placed on the instrument head 200 to detect attachment of the imaging device to the instrument head. In further examples, operation 1102 can include using a secondary camera (e.g., the detector 408) to read the machine-readable label placed on the instrument head 200.

In some further examples, the method 1100 can include preventing unauthorized use of the imaging device 400, 400b, such as by preventing image capture when attachment to the instrument head 200 is not detected in operation 1102. For example, imaging device 400, 400b is unlocked or unblocked only when it detects attachment to the instrument head 200. This can prevent use of the imaging device 400, 400b for other purposes unrelated to capturing and displaying images from an optical viewing device 100. Additionally, this can prevent use of the imaging device 400, 400b on unauthorized optical viewing devices such as devices that do not include an identifier 220 on the instrument head 200.

As shown in FIG. 11, the method 1100 includes an operation 1104 of determining a type of the instrument head. As an illustrative example, operation 1104 can include determining that the instrument head 200 is an otoscope, ophthalmoscope, dermatoscope, or other type of optical viewing device. operation 1104 can include determining the type of the instrument head based on the machine-readable data on the instrument head 200, which can be read by the imaging device 400, 400b, in accordance with the examples describe above.

In further examples, operation 1104 can include determining the type of the instrument head based on images captured by the camera 410. For example, different instrument heads (e.g., otoscope, ophthalmoscope, dermatoscope, etc.) have different fields of view such that software implemented on the imaging device 400, 400b can determine the type of the instrument head 200 attached to the imaging device 400, 400b based on a size and/or shape of the images captured by the camera 410. In some examples, the optics of the different instrument heads are modified such as to include notches, marks, labels and the like to facilitate determining the type of the instrument head based on the images captured by the camera 410 of the imaging device 400, 400b, without affecting optical performance of the instrument head 200.

When operation 1104 determines the instrument head 200 is an otoscope, the method 1100 proceeds to an operation 1106 of adjusting at least one feature of the imaging device 400, 400b for optimal use of the imaging device 400, 400b when attached to the otoscope. For example, operation 1106 can include adjusting at least one of an image size displayed on the display screen 404, a magnification of the camera 410, and a user interface displayed on the display screen 404. In further examples, operation 1106 can include displaying a workflow on the display screen 404 that is specialized for capturing images for an ear exam.

As another example, operation 1106 can include displaying a workflow that can include automatic ear detection based on identifying a location of an ear drum or other anatomy of the ear. In some examples, the workflow automatically captures an image of the ear (without user input) when the workflow detects the ear drum or other anatomy of the ear. In some examples, the imaging device 400, 400b labels anatomical structures, such as acute otitis media (AOM) or tympanic perforation, and alert the user when such structure or condition is identified.

When operation 1104 determines the instrument head 200 is an ophthalmoscope, the method 1100 proceeds to operation 1108 of adjusting at least one feature of the imaging device 400, 400b for optimal use of the imaging device 400, 400b when attached to the ophthalmoscope. For example, operation 1108 can include adjusting at least one of an image size displayed on the display screen 404, a magnification of the camera 410, and a user interface displayed on the display screen 404. In further examples, operation 1108 can include displaying a workflow on the display screen 404 that is specialized for capturing images for an eye exam.

As another example, operation 1108 can include displaying a workflow that can include automatic eye detection based on identifying a location of an optic disc or other anatomy of the eye. In some examples, the workflow automatically captures an image of the eye (without user input) when the workflow detects the optic disc or other anatomy of the eye. In some further examples, the imaging device 400, 400b can also label anatomical structures, such as papilledema or glaucomatous disc, and alert the user when such structure is identified.

When operation 1104 determines that the instrument head 200 is a dermatoscope, the method 1100 proceeds to an operation 1110 of adjusting at least one feature of the imaging device 400, 400b for optimal use of the imaging device 400, 400b when attached to the dermatoscope. The method 1100 can include additional operations for adjusting the features on the imaging device 400, 400b based on the type of instrument head determined in operation 1104. For example, operation 1110 can include adjusting at least one of an image size displayed on the display screen 404, a magnification of the camera 410, and a user interface displayed on the display screen 404. In further examples, operation 1110 can include displaying a workflow on the display screen 404 that is specialized for capturing images for a dermal exam.

As an illustrative example, operations 1106-1110 can include automatically adjusting a zoom of the camera 410 to match an optical image size of the instrument head 200 determined in operation 1104. For example, an otoscope has a smaller optical image size than an ophthalmoscope or a dermatoscope, such that operation 1106 can include increasing the zoom of the camera 410 to match the optical image size of the otoscope.

As another illustrative example, operations 1106-1110 can include centering the images displayed on the display screen 404 based on the type of instrument head determined in operation 1104. For example, images from the otoscope under higher zoom can move around the display screen 404 in an unstable manner, such that operation 1106 can include automatically centering the images to improve the usability of the imaging device 400, 400b when the imaging device 400, 400b is attached to an otoscope for examining the ears of a patient.

Operations 1106-1110 can include selecting a workflow for display on the display screen 404 based on the type of instrument head determined in operation 1104. For example, the workflow can be optimized for capturing images of one or more anatomical areas based on the type of instrument head determined in operation 1104. For example, operation 1106 can include displaying a workflow with labels for capturing images of the left and right ear drums of a patient when operation 1104 determines that the instrument head 200 is an otoscope.

As another example, operation 1108 can include displaying a workflow with labels for capturing images of the left and right eyes of a patient when operation 1104 determines that the instrument head 200 is an ophthalmoscope. In further examples, operation 1108 can include displaying one or more user interfaces associated with a workflow for capturing different types of images or information related to eye health such as eye disease diagnoses, diopter(s) selected by the diopter focus wheel 202, filter(s) selected by the filter wheel 206, and so on.

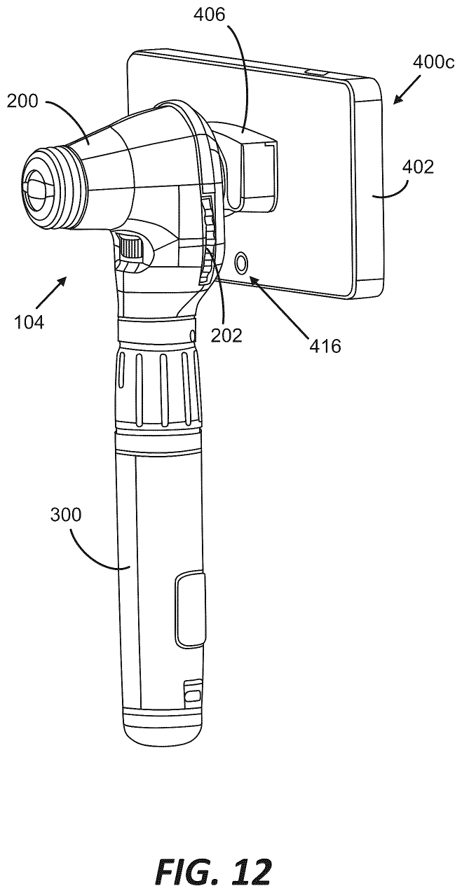

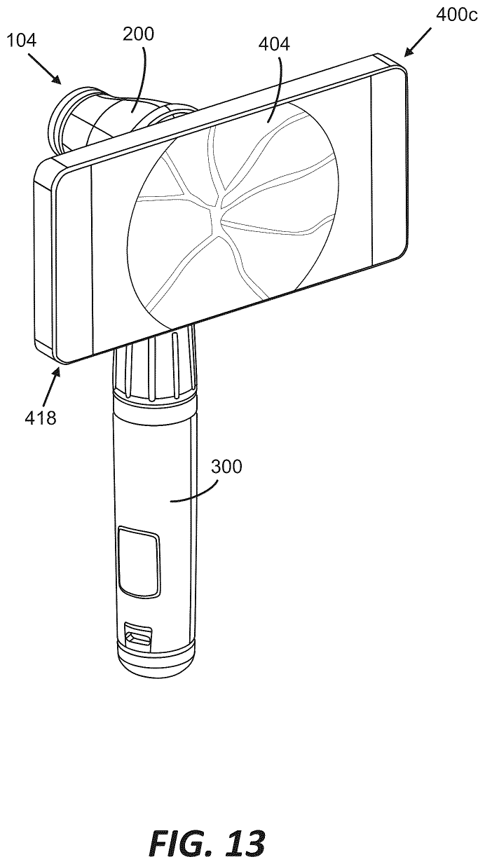

FIGS. 12 and 13 are rear isometric and front isometric views of another example of the imaging device 400c attached to the second type of optical viewing device 104. While FIGS. 12 and 13 show the imaging device 400c attached to the second type of optical viewing device 104, the imaging device 400c can similarly attach to the first and third types of optical viewing devices 102, 106, and to additional types of optical viewing devices.

The imaging device 400c is similar to the imaging devices 400, 400b shown in FIGS. 4-10. For example, the imaging device 400c includes a housing 402 having a bracket 406 for attaching to the eyepiece housing 218 of the second type of optical viewing device 104. The imaging device 400c similarly includes a display screen 404 for displaying images captured by a camera that is centrally mounted inside the housing 402 of the imaging device 400c to provide even balance and weight distribution. Like in the examples described above, the camera of the imaging device 400c is configured to align with the eyepiece 201 of the instrument head 200 for capturing and displaying images viewed through the eyepiece 201 of the instrument head 200.

In some instances, when the imaging device 400c is attached to the second type of optical viewing device 104, the housing 402 of the imaging device 400c blocks a view of the diopter readout 204 (see FIG. 2) that displays a dioptric value selected by using the diopter focus wheel 202 of the second type of optical viewing device 104. As shown in FIGS. 12 and 13, the imaging device 400c includes a mechanism 416 for displaying the dioptric value displayed in the diopter readout 204 of the instrument head 200 in a diopter readout 418 included on or proximate to the display screen 404 on the front of the imaging device 400.

In some examples, the mechanism 416 includes a secondary camera that captures an image of the dioptric value displayed in the diopter readout 204 of the instrument head 200. The image of the dioptric value is displayed in the diopter readout 418 on or proximate to the display screen 404 of the imaging device 400. As the user adjusts the dioptric value using the diopter focus wheel 202, the secondary camera captures images of the updated dioptric values for display in the diopter readout 418. In some examples, the secondary camera can also be used to read machine-readable labels such as a QR code that identifies the type of the optical viewing device such as whether the optical viewing device is an otoscope, ophthalmoscope, dermatoscope, or other type of optical viewing device.

As another example, the mechanism 416 includes a light sensor that can detect a brightness level and/or color displayed in the diopter readout 204 of the instrument head 200. Typically, in the diopter readout 204 of the instrument head 200, a dioptric value of zero (0) is displayed with a white light background, positive dioptric values (+) are displayed with a green light background, and negative dioptric values (−) are displayed with a red-light background. The white light background has a brightness level such that the light sensor can detect when the dioptric value is zero (0) based on the brightness level. Additionally, the light sensor can further detect when the dioptric value changes in a positive direction or a negative direction based on the color of light displayed in the diopter readout 204 of the instrument head 200.

The imaging device 400c can count the number of adjustments made to the dioptric value based on lens changes from turning the diopter focus wheel 202, which can be detected by the camera 410. For example, when the diopter is turned from 0 to +1 or −1 diopter, an image contrast is changed. The imaging device 400c can perform an image analysis on the contrast of the images acquired from the camera 410 to detect adjustments made to the dioptric value by turning the diopter focus wheel 202 (i.e., diopter wheel movement).

In further examples, the mechanism 416 includes a periscope that redirects light from the back surface of the housing 402 to a front surface of the housing 402 where the display screen 404 is positioned. In this manner, the periscope can be used to direct a view of the diopter readout 204 to go around the housing 402 of the imaging device 400c.

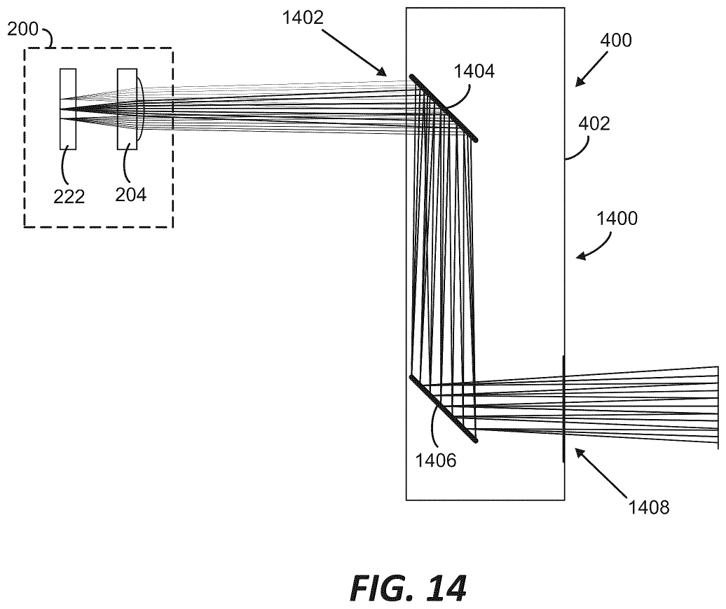

FIG. 14 is a cross-sectional view of an example of a periscope 1400 installed on the imaging device 400c in accordance with an example of the mechanism 416 described above with respect to FIGS. 12 and 13. The instrument head 200 has a diopter setting label 222 illuminated by one or more light-emitting diodes (LEDs) and magnified by the diopter readout 204. The light exits from the diopter readout 204, and enters an entrance window 1402 of the periscope.

After the light enters the entrance window 1402, a first mirror 1404 redirects the light at a 90-degree angle toward a second mirror 1406. In the example shown in FIG. 14, the first mirror 1404 is orientated at 45-degrees with respect to the entrance window 1402. The second mirror 1406 redirects the light at a 90-degree angle toward an exit window 1408 where the light exits. In the example shown in FIG. 14, the second mirror 1406 is parallel with the first mirror 1404, and is orientated at 135-degrees with respect to the exit window 1408. The periscope 1400 can include one or more lenses between or outside of the first and second mirrors 1404, 1406 to relay a view of the diopter readout 204 to go around the housing 402 of the imaging device 400c.

The exit window 1408 can be located on a corner of the display screen 404, or can be located outside of the display area of the display screen 404. In some examples, the exit window 1408 is a pinhole that displays the diopter readout 204 of the instrument head 200.

In an alternative example, the periscope can include a prism with two reflection surfaces. Unlike the example of the periscope 1400 shown in FIG. 14, where there is air space between the first and second mirrors 1404, 1406, the space between the two reflection surfaces of the prism is filled with glass or plastic (i.e., the prism is solid).

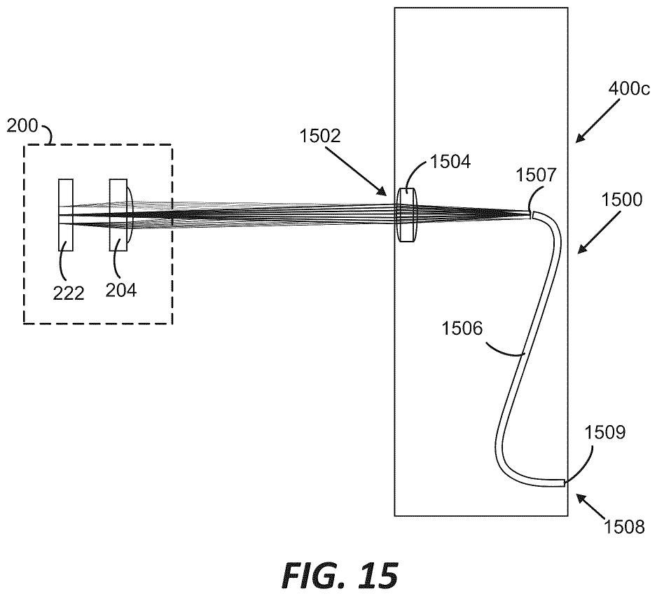

FIG. 15 is a cross-sectional view of another example of a periscope 1500 installed on the imaging device 400c. In this example, the periscope 1500 includes an entrance window 1502, a lens 1504, a fiber bundle 1506, and an exit window 1508. The lens 1504 forms an intermediate image of the diopter readout 204 on an input surface 1507 of the fiber bundle 1506. The fibers of the fiber bundle 1506 maintain a minimum resolution (e.g., enough to read the dioptric value displayed in the diopter readout 204). The dioptric value can be read directly from an output surface 1509 of the fiber bundle 1506, or a lens can be positioned over the exit window 1508 to magnify the dioptric value from the diopter readout 204 of the instrument head 200.

In further alternative examples, the housing 402 of the imaging device 400, 400b, 400c can be shaped and sized such that it does not block the diopter readout 204 on the instrument head 200. For example, the housing 402 can have a height that is less than 60 mm such that the diopter readout 204 on the instrument head 200 is not obscured.

FIG. 16 illustrates an exemplary architecture of a computing device 1600 of the imaging device 400, 400b. The computing device 1600 is used to execute the functionality of the imaging device 400 described herein. The imaging device 400 can include all or some of the elements described with reference to FIG. 16, with or without additional elements.

The computing device 1600 includes at least one processing device 1602. Examples of the at least one processing device 1602 can include central processing units (CPUs), digital signal processors, field-programmable gate arrays, and other types of electronic computing circuits. The at least one processing device 1602 can be part of a processing circuitry having a memory for storing instructions which, when executed by the processing circuitry, cause the processing circuitry to perform the functionalities described herein.

The computing device 1600 also includes a system memory 1604, and a system bus 1606 that couples various system components including the system memory 1604 to the at least one processing device 1602. The system bus 1606 can include any type of bus structure including a memory bus, or memory controller, a peripheral bus, and a local bus.

The system memory 1604 may include a read only memory (ROM) 1608 and a random-access memory (RAM) 1610. An input/output system containing routines to transfer information within the computing device 1600, such as during start up, can be stored in the read only memory (ROM) 1608. The system memory 1604 can be housed inside the housing 402.

The computing device 1600 can further include a secondary storage device 1614 for storing digital data. The secondary storage device 1614 is connected to the system bus 1606 by a secondary storage interface 1616. The secondary storage devices and their computer-readable media provide nonvolatile storage of computer-readable instructions (including application programs and program devices), data structures, and other data for the computing device 1600.

A number of program devices can be stored in secondary storage device 1614 or the system memory 1604, including an operating system 1618, one or more application programs 1620, other program devices 1622, and program data 1624. The system memory 1604 and the secondary storage device 1614 are examples of computer-readable data storage devices.

The computing device 1600 can include one or more input devices such as the display screen 404 (in examples where the display screen 404 is a touch sensitive touchscreen), one or more physical push buttons on the housing 402 of the imaging device 400, and the camera 410. Additional examples of input devices include a microphone 1626, and an accelerometer 1628 for image orientation on the display screen 404. The computing device 1600 can also include output devices such as the display screen 404, and a speaker 1630.

The input and output devices are connected to the at least one processing device 1602 through an input/output interface 1638 coupled to the system bus 1606. The input and output devices can be connected by any number of input/output interfaces, such as a parallel port, serial port, game port, or a universal serial bus. Wireless communication between the input and output devices and the input/output interface 1638 is possible as well, and can include Wi-Fi, Bluetooth, infrared, 802.11a/b/g/n, cellular, or other wireless communications.

In some examples, the display screen 404 is touch sensitive and is connected to the system bus 1606 via an interface, such as a video adapter 1642. The display screen 404 includes touch sensors for receiving input from a user when the user touches the display. Such sensors can be capacitive sensors, pressure sensors, or other touch sensors. The sensors detect contact with the display, and also the location and movement of the contact over time. For example, a user can move a finger or stylus across the display screen 404 to provide inputs.

The computing device 1600 further includes a communication device 1646 configured to establish communication across a network 1652. In some examples, when used in a local area networking environment or a wide area networking environment (such as the Internet), the computing device 1600 is typically connected to the network 1652 through a network interface, such as a wireless network interface 1650. The wireless network interface 1650 can provide Wi-Fi functionality such as for image and video transferring, live streaming, and providing a mobile hotspot. In some further examples, the wireless network interface 1650 can provide Bluetooth connectivity. Other possible examples using other wired and/or wireless communications are possible. For example, the computing device 1600 can include an Ethernet network interface, or a modem for communicating across the network.

In further examples, the communication device 1646 provides short-range wireless communication. The short-range wireless communication can include one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, a Wi-Fi technology, or similar wireless technologies.

The computing device 1600 typically includes at least some form of computer-readable media. Computer-readable media includes any available media that can be accessed by the computing device 1600. By way of example, computer-readable media can include computer-readable storage media and computer-readable communication media.

Computer-readable storage media includes volatile and nonvolatile, removable, and non-removable media implemented in any device configured to store information such as computer-readable instructions, data structures, program devices, or other data. Computer-readable storage media includes, but is not limited to, random access memory, read only memory, electrically erasable programmable read only memory, flash memory or other memory technology, or any other medium that can be used to store the desired information and that can be accessed by the computing device 1600.

Computer-readable communication media embodies computer-readable instructions, data structures, program devices or other data in a modulated data signal such as a carrier wave or other transport mechanism and includes any information delivery media. Modulated data signal refers to a signal having one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are also included within the scope of computer-readable media.

The computing device 1600 is an example of programmable electronics, which may include one or more computing devices, and when multiple computing devices are included, such computing devices can be coupled together with a suitable data communication network so as to collectively perform the various functions, methods, or operations disclosed herein.

The computing device 1600 can include a location identification device 1648. The location identification device 1648 is configured to identify the location or geolocation of the computing device 1600. The location identification device 1648 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

The various embodiments described above are provided by way of illustration only and should not be construed to be limiting in any way. Various modifications can be made to the embodiments described above without departing from the true spirit and scope of the disclosure.

What is claimed is:

1. An imaging device for capturing images viewed from an optical viewing device, the imaging device comprising:
a housing for attachment to the optical viewing device;
at least one processing device housed inside the housing; and
at least one computer-readable data storage device storing software instructions that, when executed by the at least one processing device, cause the at least one processing device to:
detect attachment to the optical viewing device;
determine a type of the optical viewing device; and
adjust at least one aspect of the imaging device based on the type of the optical viewing device.

2. The imaging device of claim 1, further comprising:
a camera for capturing the images through an eyepiece of the optical viewing device; and
a display screen for displaying the images captured by the camera.

3. The imaging device of claim 2, wherein the instructions, when executed by the at least one processing device, further cause the at least one processing device to:
determine the type of the optical viewing device based on the images.

4. The imaging device of claim 2, wherein the instructions, when executed by the at least one processing device, further cause the at least one processing device to:
adjust a zoom of the camera to match an optical image size associated with the type of the optical viewing device.

5. The imaging device of claim 2, wherein adjust the at least one feature includes centering the images displayed on the display screen based on the type of instrument head.

6. The imaging device of claim 2, wherein adjust the at least one feature includes selecting a workflow for display on the display screen based on the type of instrument head.

7. The imaging device of claim 2, wherein the instructions, when executed by the at least one processing device, further cause the at least one processing device to:
detect a diopter value selected on the optical viewing device based on the images captured by the camera; and
display the diopter value on the display screen.

8. The imaging device of claim 2, further comprising:
at least one of a secondary camera, a light sensor, and a periscope for displaying a diopter value selected on the optical viewing device.

9. The imaging device of claim 1, wherein the instructions, when executed by the at least one processing device, further cause the at least one processing device to:
prevent image capture when attachment to the optical viewing device is not detected.

10. The imaging device of claim 1, wherein the type of the optical viewing device includes an otoscope, an ophthalmoscope, or a dermatoscope.

11. A method of capturing images from an optical viewing device, the method comprising:
detecting attachment to the optical viewing device;
determining a type of the optical viewing device; and
adjusting at least one aspect based on the type of the optical viewing device.

12. The method of claim 11, further comprising:
detecting the type of the optical viewing device based on the images.

13. The method of claim 11, further comprising:
detecting the type of the optical viewing device based on a wireless signal received from the optical viewing device.

14. The method of claim 11, further comprising:
adjusting a camera zoom to match an optical image size associated with the type of the optical viewing device.

15. The method of claim 11, further comprising:
centering the images on a display screen based on the type of the optical viewing device.

16. The method of claim 11, further comprising:
selecting a workflow based on the type of the optical viewing device.

17. The method of claim 11, further comprising:
preventing image capture when attachment of the optical viewing device is not detected.

18. The method of claim 11, further comprising:
detecting a diopter value selected on the optical viewing device; and
displaying the diopter value.

19. The method of claim 11, further comprising:
using at least one of a camera, a light sensor, and a periscope for displaying a diopter value selected on the optical viewing device.

20. An imaging device for capturing images viewed from an optical viewing device, the imaging device comprising:
a housing having a bracket for attaching the imaging device to the optical viewing device;
a camera for capturing the images through an eyepiece of the optical viewing device;
a display screen for displaying the images captured by the camera;
at least one processing device; and
at least one computer-readable data storage device storing software instructions that, when executed by the at least one processing device, cause the at least one processing device to:
display a diopter value on the display screen, the diopter value selected on the optical viewing device.
